(19) United States
(12) Patent Application Publication
(10) Pub. No.: US 2007/0055175 A1
(43) Pub. Date: Mar. 8, 2007

(54) Devices and methods for tissue analysis

(75) Inventor: Richard G. Caro, San Francisco, CA (US)

Correspondence Address: LADAS & PARRY, 26 WEST 61ST STREET, NEW YORK, NY, 10023, US

(73) Assignee: PULMOSONIX PTY LTD

(21) Appl. No.: 11/138,660

(22) Filed: May 25, 2005

(51) Int. Cl. A61B 5/103; A61B 5/117

(52) U.S. Cl. 600/587

(57) ABSTRACT

Devices and methods are provided for assessing the condition of tissue, in particular to diagnose the location and stage of diseases such as emphysema. In one device or method according to the invention, sound is introduced into lung tissue. A portion of the sound is detected after it has passed through a portion of the lung. A sound-propagation parameter is determined by comparing a property of the introduced sound and a property of the detected sound. A region of examination of the tissue is identified, based in part on either a property of the component that introduces the sound or a property of the component that detects the sound. The condition of the tissue is assessed in the region of examination, based in part on the sound-propagation parameter.

### BACKGROUND OF THE INVENTION

1. Field of the Invention

This invention relates assessing the condition of tissue, and more particularly to assessing whether and to what extent specific regions of the lung are affected by disease.

2. Background Information

Determination of the condition of biological tissues without biopsy is useful in many circumstances: for example, when the region of tissue to be examined is inaccessible, or when the process of biopsy can cause pain or create medical complications, or be otherwise undesirable.

Techniques presently used in determining the characteristics of biological tissues include x-rays, magnetic resonance imaging (MRI) and radio-isotopic imaging. These are generally expensive and involve some degree of risk which is usually associated with the use of x-rays, radioactive materials or gamma-ray emission. Furthermore, these techniques are generally complicated and require equipment which is bulky and expensive to install and, in most cases, cannot be taken to the bedside to assess biological tissues in patients whose illness prevents them being moved.

Sound waves, particularly in the ultra-sound range have been used to monitor and observe the condition of patients or of selected tissues, such as the placenta or fetus. However, the process requires sophisticated and sometimes expensive technology and cannot be used in tissues in which there is a substantial quantity of gas, such as the lung.

The lungs supply oxygen to, and remove carbon dioxide from, the blood. Air enters the lungs via the trachea and the bronchial tube of each lung. The two bronchial tubes branch into secondary bronchi that form the lobes of the lung, and these secondary bronchi further branch to form numerous smaller tubes (bronchioles) that terminate in small gas-exchanging air sacs called alveoli. A network of capillaries runs through the walls of the alveoli, and oxygen and carbon dioxide are exchanged across these walls between the air in the alveoli and the blood in the capillaries.

Relating to the condition of the lung, Chronic Obstructive Pulmonary Disease (COPD) is the leading cause of respiratory deaths worldwide. About three million patients in the US have emphysema, one form of COPD. COPD places enormous economic burden on society. Medical expenses for COPD patients are extremely high because of frequent visits to the emergency room, extended hospital stays and expensive medications. In developed countries, the major cause of COPD is cigarette smoking but two distinct and overlapping diseases (together called COPD) result: chronic bronchitis and emphysema.

Chronic bronchitis is a neutrophil led chronic inflammatory airways disease with regular exacerbations leading to true narrowing of airways and increased resistive work of breathing. The key elements of therapy are the removal of the toxic stimulus (i.e., smoking cessation), bronchodilator therapy, anti-inflammatory drugs, mucolytics, prevention and early treatment of infection as well as rehabilitation.

In emphysema, the problems are distinctly different. The lung parenchyma is destroyed with a reduction in gas exchanging area. Dynamic collapse of untethered airways occurs leading to increased expiratory work of breathing and gas trapping of the lung. This gas trapping makes the lung work at a higher lung volume (at which it is stiffer), increasing inspiratory work of breathing. The over-distension also markedly reduces the mechanical efficiency of the diaphragm. Exercise is terminated early because of rapidly rising and unsustainable work of breathing.

Smoking cessation and prevention of infection are the keys to prevent disease progression. Bronchodilators and anti-inflammatory drugs would be predicted to have little benefit but rehabilitation is of proven benefit. In more advanced disease interventions to improve the mechanical properties of the lung, for example lung volume reduction surgery and highly novel minimally invasive approaches, as well as transplantation in a few, are the most likely to significantly improve functional capacity.

Emphysema is a slowly progressive disease of the lung. It involves the gradual destruction of the alveolar walls. The loss of alveolar tissue results in a loss of gas exchange surface area and decreases the number of capillaries available for gas exchange. It also reduces the elastic recoil of the lung and leads to the collapse of the bronchioles and chronic airflow obstruction. Thus, lung function is gradually lost through a reduction in gas-exchange area and in the amount of air that reaches the alveoli.

Emphysema afflicts millions of people worldwide. Statistics show that in 2002 over three million people were affected by the disease in the US alone, 50% being over 65 years of age. By 2020, emphysema and obstructive airway disease are expected to be the third leading cause of death after cancer and heart disease. Although the exact causes of the disease are not understood, smoking is a major factor, with an estimated 20% of smokers contracting the disease at some time in their lives.

Advanced emphysema is very debilitating, but certain types of surgical intervention have been shown to benefit patients. In particular, lung volume reduction surgery (LVRS) for certain patients has been shown benefits regarding symptomatic improvement and physiological response. (See Brenner et al., Chest, 126 (1) July 2004, pp. 239-248, and references therein.)

There is growing interest in performing such procedures through noninvasive or minimally invasive approaches (e.g., via catheters, bronchoscopes, and the like inserted through the throat or through small chest incisions.) Some of these are reviewed by Brenner et al. in the above-cited reference.

Research has also been undertaken to develop minimally invasive tools to perform these types of surgery, and a new field of interventional pulmonology is emerging. Many of the proposed procedures that have thus far emerged from the research involve surgical intervention to alter the properties of a portion of the lung. For example, surgery can collapse or excise a portion of the lung, or bronchial valves can be used to isolate and collapse a portion of the lung, or both techniques may be used. Other therapies, such as the use of glue to collapse lung portions, are also being discussed. Another field of research involves the use of stem cell therapies to induce regeneration of certain portions of lung tissue.

Whatever method is used, it is important to be able to correctly identify the portion of the lung to be treated, and therefore it is desirable to have a technique to identify which portions of the lung have disease, and to be able to stage that disease. Ideally clinicians would be able to determine where in the lung the disease was located, the nature of the disease, and its severity.

Currently, CT-xray techniques allow lung images to be created that provide some information about location and extent of disease. There are also lung diffusion tests and the technique of spirometry, which provides information about overall lung function. These methods are however somewhat complex and expensive, and are not suitable for intraoperative use, and are limited in resolution.

The techniques of bronchoscopy and endobronchical ultrasound can provide information about the interior surface of the lung and tissue in a specific site, but are limited to providing information on material at most a small distance below the surface.

It would be very desirable to have a method and apparatus that could be used during surgical intervention in advanced stage emphysema patients, and which could provide information to the physician about the nature of the lung in a specific region near which he or she is planning an intervention. It would also be valuable if this technique could provide information about tissue deep in the lung, where the resolution of techniques such as CT scanning is insufficient to provide localized information about the existence, stage, and nature of the disease and of the lung condition.

Others have described a noninvasive method and apparatus for detecting emphysema in the lung using sound propagation through the lung. However, that technique does not provide a means for learning about tissue in a specific location associated with an interventional device such as a bronchoscope or catheter. Nor does it allow very well defined spatial localization of the information obtained.

It is desirable to have methods and devices that are noninvasive (or minimally invasive) and that address the shortcomings in existing methods and devices.

### SUMMARY OF THE INVENTION

Devices and methods are provided for assessing the condition of tissue. In one method according to the invention, the steps include:
introducing sound into tissue with a sound-introducing means;
detecting a portion of the sound with a sound-detecting means after it has passed through at least a portion of the tissue;
determining at least one sound-propagation parameter by comparing at least one property of the introduced sound and at least one property of the detected sound;
identifying a region of examination of the tissue, based in part on either at least one property of the sound-introducing means or at least one property of the sound-detecting means; and
assessing the condition of the tissue in the region of examination, based in part on the sound-propagation parameter.

In one device according to the present invention, the device includes:
means for introducing sound into tissue;
means for detecting a portion of the sound after it has passed through at least a portion of the tissue;
means for determining at least one sound-propagation parameter by comparing at least one property of the introduced sound and at least one property of the detected sound;
a means for identifying a region of examination of the tissue, based in part on either at least one property of the sound-introducing means or at least one property of the sound-detecting means; and
means for assessing the condition of the tissue in the region of examination, based in part on the sound-propagation parameter.

In other methods and devices according to the invention:
the introduced sound has the majority of its energy within a frequency range from about 100 Hz to about 50 kHz;
the introduced sound has the majority of its energy within audible frequencies;
the tissue is lung tissue;
the introduced sound is selected may be: a tone, a pulse, white noise, pseudorandom noise, a sequence of tones, a complex multifrequency waveform, a swept frequency signal, a frequency-modulated signal, or an amplitude-modulated signal;
the at least one sound-propagation parameter may be: phase delay, phase velocity, group velocity, amplitude, relative amplitude, attenuation, dispersion, the first derivative of amplitude as a function of frequency, or the ratio of amplitude A1/A2, where A1 is a first sound amplitude in one frequency band, and A2 is a second sound amplitude in a second frequency band;
the at least one property of the introduced sound may be: phase, amplitude, velocity, power, energy, or frequency;
the at least one property of the detected sound may be: phase, amplitude, velocity, power, energy, or frequency;
the at least one property of the sound-introducing means may be: spatial location, size, orientation, or shape;
the at least one property of the sound-detecting means may be: spatial location, size, orientation, or shape;
assessing the condition of the tissue includes first calculating a tissue-condition parameter;
introducing sound includes inserting the sound-introducing means within the tissue;
detecting a portion of the sound includes inserting the sound-detecting means within the tissue;
the sound-introducing means is located within the tissue;
the sound-detecting means is located within the tissue;
the region of examination is in the vicinity of an interventional or diagnostic device that has been positioned within or adjacent to the tissue;
the interventional or diagnostic device may be: a bronchoscope, a catheter, or an endoscope;
the sound-detecting means is attached to the interventional or diagnostic device;
the sound-introducing means is attached to the interventional or diagnostic device;
the interventional or diagnostic device includes a reflective device that reflects the introduced sound in the general direction of the sound-detecting means;
the reflective device is attached to the interventional or diagnostic device;
the tissue-condition parameter is an index indicative of: tissue microstructure, airway dimensions, fenestrae size, airway conductance, tissue permeability, tissue permittivity, tissue elasticity, or tissue viscosity; or
information is derived relating to the type or stage of disease in the tissue by comparing the tissue-condition parameter with a library of tissue-condition parameters derived from clinical studies.

In another device according to the invention, the device includes:
a first transducer that introduces sound into tissue;
a second transducer that detects a portion of the sound after it has passed through at least a portion of the tissue;
a sound-propagation comparator that determines at least one sound-propagation parameter by comparing at least one property of the introduced sound and at least one property of the detected sound;
an examination-region identifier that identifies a region of the tissue, based in part on either at least one property of the first transducer or at least one property of the second transducer; and
a tissue-condition assessor that assesses the condition of the tissue in the region of examination, based in part on the sound-propagation parameter.

In other devices according to the invention:
the at least one property of the first transducer may be: spatial location, size, orientation, or shape;
the at least one property of the second transducer may be: spatial location, size, orientation, and shape;
the tissue-condition assessor comprises a parameter calculator that calculates a tissue-condition parameter;
the first transducer is inserted within the tissue;
the second transducer is inserted within the tissue;
the first transducer is located within the tissue;
the second transducer is located within the tissue;
the first transducer is attached to the interventional or diagnostic device;
the second transducer is attached to the interventional or diagnostic device;
the interventional or diagnostic device comprises a reflective device that reflects the introduced sound in the general direction of the second transducer; or
an information deriver that derives information relating to the type or stage of disease in the tissue by comparing the tissue-condition parameter with a library of tissue-condition parameters derived from clinical studies.

### BRIEF DESCRIPTION OF THE DRAWING(S)

FIG. 1 is a schematic diagram showing one preferred embodiment of the invention.

FIG. 2 is a flow chart illustrating one method of analyzing the signals that may be used with the invention.

FIG. 3 shows a schematic diagram indicating the theory behind the detection method of an embodiment of the present invention.

FIG. 4 is a graph of signal frequency against velocity through a lung for various sizes of fenestrae.

FIG. 5 is a graph of signal frequency against velocity through the lung for a number of lung analogs exhibiting different sizes of fenestrae.

### DETAILED DESCRIPTION OF THE PREFERRED EMBODIMENTS

Methods and devices according to preferred embodiments of the present invention perform a localized measurement of the properties of tissue, such as lung tissue, with a view to determining whether or not the tissue is diseased, such as emphysematous or affected by cystic fibrosis, and the degree to which the disease has progressed. Additional results of this measurement would be a diagnosis of the disease stage of that portion of an organ, such as a lung.

The present description will primarily address preferred embodiments of methods and devices according to the invention in which lung tissue is assessed for the presence and stage of emphysema, or cystic fibrosis or chronic bronchitis. But as described in the previous paragraph, other methods and devices according the invention can assess the presence and stage of other diseases in other tissue or in other organs or portions of organs.

Characteristics of biological tissues can be determined by measuring the velocity and attenuation of a sound as it propagates through the tissue. This can be achieved by introducing a sound to a particular location or position on the tissue, allowing the sound to propagate through the tissue and measuring the velocity and/or attenuation with which the sound travels from its source to its destination, the destination including a receiver which is spatially separated from the sound source.

It is particularly desirable that the tissue is porous comprising a composite structure made up of tissue and gas, or has regions of high and low density. Preferably the tissue is of the respiratory system. More preferably the tissue is lung tissue.

Commercially available acoustic hardware and software packages may be used to generate a psuedo-random noise or other acoustic signal, and to perform initial data processing. External noise which is not introduced to the tissue as part of the psuedo-random noise signal is strongly suppressed by cross-correlation thereby improving the quality of the measurements made.

A separate analysis of the relative transmission of the sound through the tissue can be used to identify resonant and anti-resonant frequencies of the thorax and tissue which is being assessed. Changes in these frequencies can then be used to assess regional differences in tissue topology which may be related to pathology.

Using relationships between sound transmission velocity in tissues and the tissue characteristics themselves, it is possible to obtain a workable relationship between acoustic measurements and lung pathology.

The invention uses devices and methods that provide a virtually continuous real-time determination of disease state or tissue characteristics by monitoring acoustic transmission characteristics such as velocity and or attenuation of a sound signal as it propagates through the lung. Devices and methods according to the invention are applicable in both adults and infants, and for humans and animals.

Since lung disease often manifests in reduced lung volume, a comparison can be used, again, to provide an indication as to whether a subject's lung exhibits a propensity for lung disease. Common lung diseases may include emphysema, asthma, regional collapse (atelectasis), interstitial edema and both focal lung disease (e.g. tumor) and global lung disease (e.g. emphysema). Each of these may be detectable when measurements of parameters such as but not limited to the velocity and or attenuation of a sound which is transmitted through a diseased lung is compared with that of a lung in normal condition.

In addition, spectral analysis of the impulse response can indicate frequency components in the sound signal which are more prominent than others and which may be an indicator of pathological or abnormal tissue.

The etiology of COPD is not well understood. However, in accordance with embodiments of the present invention, the acoustic transmission characteristics of a lung are determined, and analyzed to determine if they are indicative of a feature of COPD such as fenestrae in the alveoli, inflammation of the bronchial tubes, bronchorestriction, or increased mucus production in the airways. In emphysema, a special form of COPD, perforations (fenestrae) appear in the walls of the alveoli, changing the structure of the lung from a closed-cell type tissue structure towards an open-cell tissue structure. This causes the lung to lose elasticity and eventually leads to a collapse of associated bronchioles.

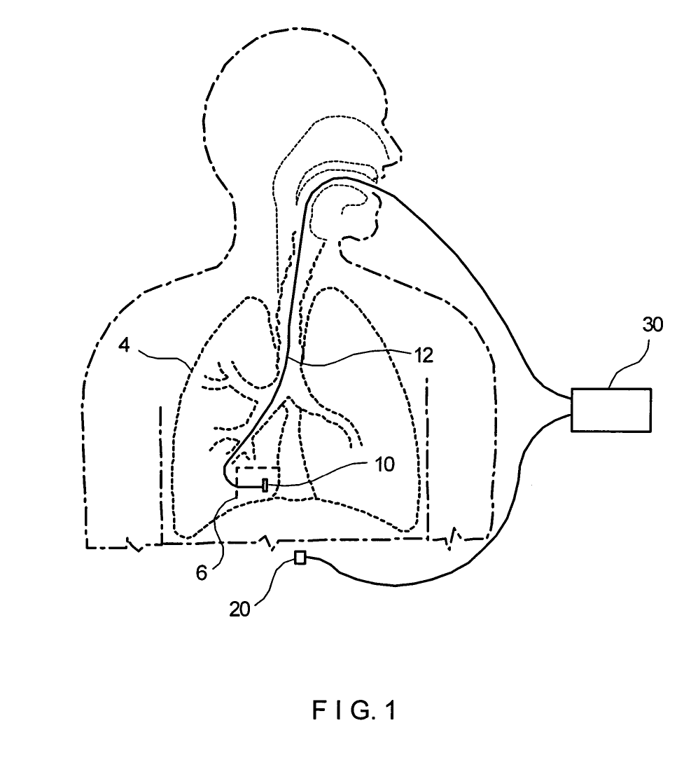

FIG. 1 depicts one embodiment of the invention as used to assess the condition of lung tissue. Tone transmitter 10, which can generate sound as a tone, is inserted into lung portion 6 of lung 4—one localized region of examination—using bronchoscope 12.

As those skilled in the art will recognize, tone transmitter 10 is only one example of a sound-introducing means. Other sound-introducing means that may be used include loudspeakers, electromagnetic vibrators, piezoelectric actuators, or other means for transducing vibration or sound, and any other transducer. A suitable sound-introducing means may produce sound or other acoustic energy in any form, including a pulse, white noise, pseudorandom noise, a sequence of tones, a complex multifrequency waveform, a swept frequency signal, a frequency-modulated signal, and an amplitude-modulated signal.

Similarly, skilled artisans will recognize that bronchoscope 12 is only one example of an insertion means. Other insertion means that may be used include a catheter, an endoscope, and any other interventional or diagnostic device.

Tone transmitter 10 introduces sound into tissue in lung portion 6. Preferably the introduced sound has the majority of its energy within a frequency range from about 100 Hz to about 50 kHz. In another preferred embodiment, the introduced sound has the majority of its energy within the audible frequency range. Those skilled in the art will recognize other frequencies or frequency ranges that may be used.

The acoustic signal should have sufficient amplitude to produce an acceptable signal-to-noise ratio. An example of a suitable sound pressure level applied to the thorax is 120 decibels or approximately 20 Pascals though other levels may also be suitable. It should be noted that, since the signal is applied directly to a small area of the body, high decibel signals can be used without discomfort, as the transducer is sufficiently shielded that the sound is barely audible to the subject.

The acoustic signal may include frequencies in the range of 70 Hz to 5 kHz, as these frequencies have been found to produce very good results. In one embodiment, frequencies lower than 1 kHz are used.

As the sound introduced by tone transmitter 10 passes through the tissue of lung portion 6, part of the sound is absorbed and part of the sound passes through the tissue. Tone sensor 20 is placed outside the patient's body, in a position to detect the part of the introduced sound after it passes through the tissue. As those skilled in the art will recognize, tone sensor 20 is only one example of a sound-detecting means. Other sound-detecting means that may be used include microphones of various types, hydrophones, other devices for converting sound or vibration to electrical signals, and any other transducer.

Preferably, tone sensor 20 is adjacent to the portion of bronchoscope 12 that is positioned within lung portion 6, the region of examination in this example. As skilled artisans will appreciate, tone transmitter 10, or any sound-introducing means, may be placed outside the patient's body, and tone sensor 20, or any sound-detecting means, may be positioned within lung portion 6.

Preferably, when tone transmitter 10 or tone sensor 20 is positioned within the lung, the one of those that is so positioned is closely coupled to the lung tissue. One example for accomplishing this is to use a saline inflated balloon, such as is commonly used in intrabronchial ultrasound. This balloon could have a variety of shapes and need not occlude the patient's airway, which provides air to the lungs.

Other preferred embodiments may use an array of tone transmitters 10 (or other sound-introducing means), or an array of tone sensors 20 (or other sound-detecting means), or arrays of both. Any such arrays may be used in accordance with the invention to assess single or multiple parts of the lung (or other organs) contemporaneously.

In another preferred embodiment, a reflective device is attached to bronchoscope 12 that reflects the introduced sound in the general direction of tone sensor 20.

Importantly, in each of these preferred embodiments the user, or perhaps a piece of software in the processor, is aware of the locations of the tone sensor and the sound detecting means, and perhaps their orientation. Because the sound travels from one sensor to the other and one of them is located in close proximity with a bronchoscope, or other similar device, the measurements of tissue properties will be associated with that specific region of tissue positioned in the region between the tone sensor and the detector, and adjacent to the bronchoscope. This has the great advantage that it provides information specifically about that region of tissue that sits in the neighbourhood of the bronchoscope. This would not be the case in alternate methods in which the sound is injected at another location, and the detectors are located along the chest wall, and none of them are physically or spatially connected with the bronchoscope.

There are several additional features of the preferred embodiments that facilitate localization of the measurement.

The sound propagates across the lung rather than along it as is commonly described in the literature, and because of the propagation patterns of the sound, it is possible to model quite well the region of tissue that predominantly affects signal propagation, and to localize it to a quite small volume.

The sound wavelength may be relatively large compared to the spatial dimensions of interest within the tissue. In the geometry described in the preferred embodiments it is possible to select a detector dimension so that the path being sampled by the detected sound is determined primarily by detector dimensions rather than by sound wavelength.

It may also be possible to choose specific detector or tone sensor orientations to modify the path through the tissue, thereby further constraining or localizing the region of tissue being sampled.

In the example of FIG. 1, control unit 30 is in communication with tone transmitter 10 and tone sensor 20. Preferably, control unit 30 is structured and operates as follows.

Control unit 30 includes a computer processor, memory, and data storage. One example of control unit 30 is a personal computer. Control unit 30:
sends a sound signal to transmitter 10, which transmitter 10 uses to generate the introduced sound;
receives a sound signal that tone sensor 20 produces when detecting the portion of the introduced sound after it passes through the tissue of lung portion 6; and
processes the sent and received sound signals to determine the presence and stage of any emphysema in lung portion 6.

The processing that control unit 30 does includes:
determining at least one sound-propagation parameter (e.g., phase delay) by comparing at least one property of the introduced sound (e.g., phase) and at least one property of the detected sound (e.g., phase);
identifying the region of examination of the tissue (e.g., lung portion 6) based in part on either at least one property of tone transmitter 10 (e.g., spatial location) or at least one property of the tone sensor 20 (e.g., spatial location); and
assessing the condition of the tissue in the region of examination, based in part on the sound-propagation parameter (e.g., phase delay).

For the processing described above:
other sound-propagation parameters that may be used include: phase velocity, group velocity, amplitude, relative amplitude, attenuation, dispersion, the first derivative of amplitude as a function of frequency, and the ratio of two amplitudes A1 and A2 (i.e., A1/A2), where A1 is a first sound amplitude in one frequency band, and A2 is a second sound amplitude in a second frequency band;
other properties of the introduced sound that may be used include: amplitude, velocity, power, energy, and frequency;
other properties the detected sound that may be used include: amplitude, velocity, power, energy, and frequency;
the properties of the introduced sound and the detected sound used may be the same or different;
other properties of tone transmitter 10 that may be used include: size, orientation, and shape;
other properties of tone sensor 20 that may be used include: size, orientation, and shape; and
the properties of tone transmitter 10 and tone sensor 20 used may be the same or different.

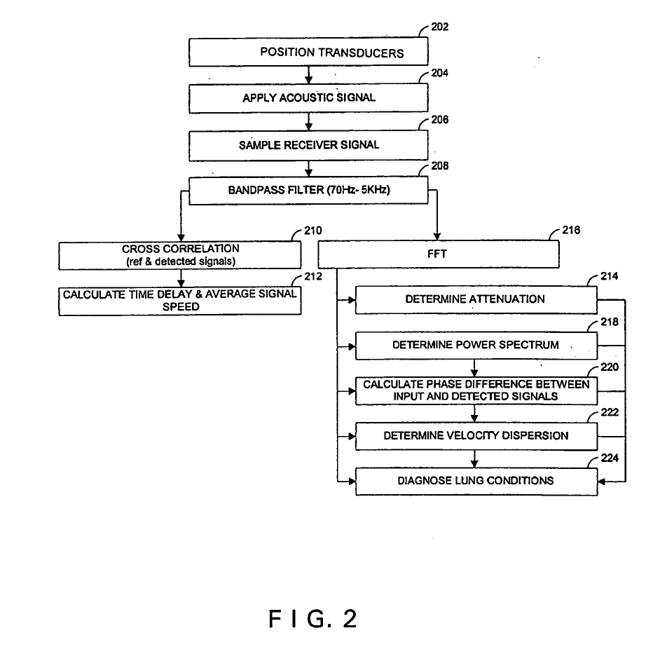

FIG. 2 is a flow chart showing one method for analyzing signals that may be used with systems and methods according to the invention to determine the presence of COPD in a lung. In a step 202, transducers (such as tone transmitter 10 and tone sensor 20) are positioned inside and outside a patient's body in accordance with the invention. In a step 204, an acoustic signal is applied to the lung. In a step 206, the signal is detected after it has passed through at least part of the lung. In steps 208 to 224, one or more acoustic transmission characteristics of the lung are determined. COPD is determined to be present when an acoustic transmission characteristic, indicative of the microstructure of the lung, indicates the presence of a feature of COPD.

In another preferred embodiment, control unit 30, when processing data to assess the condition of the tissue, first calculates a tissue-condition parameter (e.g, tissue microstructure). Other tissue-condition parameters that may be used include: airway dimensions, fenestrae size, airway conductance, tissue permeability, tissue permittivity, tissue elasticity, and tissue viscosity.

Calculating the tissue-condition parameter may be done as follows.

In one embodiment, the present invention exploits the effect that fenestrae (perforations) in the alveoli of a lung have on the acoustic transmission characteristics of the signal to indicate the onset of emphysema and the progression of the disease. It recognizes that changes in the microstructure or alveolar structure of the lung caused by an increase in the number of fenestrae or pores connecting neighboring alveoli, and which may be seen as a movement from a closed-cell type arrangement to an open-cell type arrangement, will cause a measurable and identifiable change in the acoustic transmission properties of the lung.

This change in the “cellular structure” of the lung (i.e., open vs. closed) has the effect of changing the acoustic permeability of the lung tissue, which can be detected by monitoring the acoustic transmission characteristics of the lung. At least in the emphysematous lung, the changes in cell-type occur in very early stages when the patient may still be asymptomatic and before there is any noticeable change in the lung density.

In one embodiment of the present invention, signal velocity through the lung is detected, and a determination is made as to whether one or more of the detected velocity characteristics are indicative of perforated/fenestrated alveoli. Thus, a determination may be made as to whether the velocity of an acoustic signal through a lung is greater than a signal velocity associated with a normal lung. The magnitude of the signal velocity may be used to indicate the stage of emphysema, as may changes in velocity which are detected as the signal propagates through the lung.

The signal velocity may be determined for a single acoustic frequency, or for a range of frequencies. In the latter case, emphysema may be determined based on a characteristic of the velocity profile over a range of frequencies or an average of the velocities. In one embodiment, the velocity dispersion may be determined. Thus, generally for a normal or diseased lung, signal velocity will vary based on signal frequency. In accordance with embodiments of the present invention, an increase in velocity dispersion, i.e., a larger spread of velocities for a particular frequency range (or put another way a larger change in velocity for a particular frequency range) may indicate existence of COPD features such as alveolar fenestrae which are indicative of emphysema. The amount of dispersion may indicate the degree of COPD/emphysema or stage thereof.

In one embodiment, signal attenuation through the lung is detected, and a determination is made as to whether one or more of the detected attenuation characteristics are indicative of a feature of COPD such as, for example, perforated alveoli, inflammation of the airways, bronchorestriction, or increased mucus production in the airways. Thus, a determination may be made as to whether the attenuation of an acoustic signal through a lung is different from signal attenuation associated with a normal lung and indicative of COPD. The amount of the signal attenuation may be used to indicate the degree of COPD/emphysema and/or provide an indication as to the stage of development of the disease.

Attenuation may be determined for a single frequency, or for a range of frequencies. In the latter case, COPD may be determined based on a characteristic of the attenuation profile over a range of frequencies, or an average of the attenuation. In one embodiment, the frequency dependence of attenuation may be determined. Thus, generally for a normal or diseased lung, the signal attenuation will vary based on signal frequency, for example, a change in attenuation may be more noticeable for lower frequency sounds. In accordance with the present invention, a larger change in attenuation at certain frequencies may be used to indicate existence of features of COPD. The magnitude of this change may indicate the degree of COPD.

It is to be understood that a combination of two or more of the above characteristics may be used to assess the existence of emphysema. For example, the velocities of one or more of the acoustic frequencies and the velocity dispersion of the acoustic signal may both be used to assess emphysema. Similarly, a combination of two or more of the above characteristics may be used to assess the existence of chronic bronchitis or other forms of COPD. For instance, the attenuation of one or more of the acoustic frequencies and the attenuation dispersion of the acoustic signal may both be used to assess the existence of chronic bronchitis. Other acoustic characteristics such as signal power density may be used as an alternative or in addition to the above.

In another preferred embodiment, control unit 30 derives information relating to the type or stage of emphysema in the tissue of lung portion 6 by comparing the tissue-condition parameter with a library of tissue-condition parameters derived from clinical studies. Such studies typically include data that is indicative of healthy and diseased patients of various heights, weight ages, etc., and that estimates whether or not the lung is diseased and/or the extent of disease and/or the type of disease. Those having pertinent skill can conduct such studies using protocols known in the art.

There are several possible ways in which the method and apparatus described here can benefit the interventional pulmonologist. In one procedure under development, and described by Brenner et al., the pulmonologist places one way valves in the bronchi in order to collapse portions of the surrounding lung. In order to do that, regions of disease in the lung are mapped in advance using techniques such as CT xray. With the device described here the pulmonologist can examine specific portions of the lung during the placement of the valves to ensure that the valves are being placed in regions with appropriate levels of disease. In addition, the device may be passed distally into higher segments of the lung and can determine how best to treat those, and where to place interventional devices such as valves in those higher segments, based on the nature and distribution of disease determined by the method described herein. In addition, after placement of an initial valve it is important to be able to determine whether there is collateral air flow that bypasses the valve and to analyze the lung in the regions of collateral flow and intervene with additional therapies that alter that collateral flow. The method and apparatus described here can provide real time guidance as to the tissue characteristics in the surrounding region, and may also provide information about regions of collateral flow.

The input sound may be, for example, a single tone or a plurality of tones emitted simultaneously or separately. They may be emitted in bursts, and their times-of-flight and amplitudes may be recorded by the controller using phase, impulse response or other suitable determinations. In one embodiment, the input acoustic signal is a pseudorandom noise signal. The controller then cross-correlates this input signal with the signal received at the receiver, e.g., by cross-correlating the received signal with a signal produced at a receiver near the transducer or by cross-correlating it with the control signal applied to the transducer.

The cross-correlation can be used to determine the impulse response of the chest, and, by using a Fast-Fourier Transform of this response, the frequency domain transfer function can be determined. Using the FFT, the velocity, attenuation and their variation (as a function of frequency, i.e., dispersion in the case of velocity) may be determined along with the power spectrum of the lung.

When injecting a pulsed tone signal, the outputs from one (or a plurality of) receiver(s) may also be cross-correlated to establish the transit time (velocity) and amplitude of the pulse arriving at the chest wall, at the location of each of the receivers. This process can be repeated for a number of tone frequencies, and using the measurements, a parameter such as velocity dispersion, Δ, may be calculated as:

$$Δ = \frac{{v}_{2} - {v}_{1}}{{f}_{2} - {f}_{1}} ,$$

where v₁ is the sound velocity measured at frequency f₁, and v₂ is the sound velocity measured at frequency f₂.

The frequency dependence of attenuation could be calculated similarly.

These results may be used to determine the existence and stage of COPD. One particular form of COPD which is well suited to this method of detection is emphysema. The various acoustic characteristics may be compared with those of a normal lung to indicate whether there are significant differences and those differences may be taken as an indication of the presence of a feature of COPD. Also, for any particular subject or subject-type, velocity or attenuation or other variable standards may be set for indicating emphysema against which the results may then be compared.

Generally, a higher than expected velocity for a particular frequency may indicate emphysema, due to increased communication between adjacent alveoli, as may a larger than expected velocity dispersion. A higher than expected velocity for a particular frequency or range of frequencies and lower signal attenuation at higher frequencies, may indicate chronic bronchitis.

The results for the various acoustic transmission characteristics may be combined in the assessment so as to reinforce the judgment and/or so as to indicate the degree or stage of COPD. Thus, a velocity increase and a velocity dispersion increase may be used together to indicate the presence of fenestrae and so emphysema. The acoustic transmission characteristics determined using the inventive method and apparatus may also be used in combination with more traditional methods such as spirometry and x-ray methods, where further clinical support for a finding is warranted.

The degree of velocity increase, and velocity dispersion and the like may also be used to determine the degree or stage of COPD, where later stages of the disease correspond with larger fenestrae and therefore larger changes in detected signal velocities and dispersions.

The present detection methods use the knowledge that COPD and, in particular, emphysema can be detected by transmitting an acoustic signal through a lung or part thereof, and monitoring the acoustic transmission characteristics which are attributable to features of COPD such as, in the case of emphysema, a microscopic change in the structure of the alveoli. These changes include appearance of fenestrae in the onset and progression of the disease which can be determined by measurable changes in the acoustic permeability of the lung.

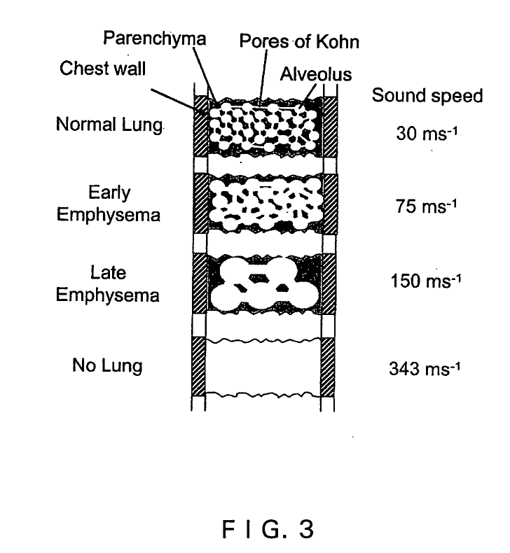

FIG. 3 shows conceptually the change in velocity characteristics with deterioration of the lung. In a normal lung, the speed of sound may be for example, 30 ms⁻¹ for one particular acoustic frequency, and increases with higher frequencies. As the lung deteriorates, however, the number of alveolar fenestrae increases, and the air sacs lose their definition and form larger sacs. As this occurs, the velocity of any particular frequency signal will increase, as shown, for example, to 75 or 150 ms⁻¹, with an extreme limit of no lung tissue (only air) producing a sound wave of 343 ms⁻¹ (which of course will not occur in practice).

From an analytical point of view, an emphysematous lung may be perceived as an elastic material including gas-containing cells that are linked with pores that grow with time as the emphysema progresses. Sound waves propagate through this environment via the pores, which cause a loss of energy via viscous and heat losses to the cell walls. The parameters that determine velocity and attenuation in this setting may be determined by using the conservation laws of mass and momentum that govern wave motion in porous media. These can be stated as follows:

$$\begin{matrix}\frac{\partial {v}_{g}}{\partial x} = - \frac{1}{{K}_{g}} \frac{\partial {p}_{g}}{\partial t} & ( 1 ) \\ \frac{\partial {p}_{g}}{\partial x} = - \frac{{ϕ}_{g} η}{{k}_{0}} {v}_{g} & ( 2 )\end{matrix}$$

where η, K_{g}, p_{g}, v_{g} are the viscosity, bulk modulus, pressure and velocity respectively, and φ_{g} is the ratio of gas volume to tissue volume (gas fraction) in the lung, and

k₀ is the permeability or the ease with which sound waves propagate through the porous lung tissue.

Differentiating (2) with respect to x gives:

$$\frac{{\partial }^{2} {p}_{g}}{\partial {x}^{2}} = \frac{{ϕ}_{g} η}{{k}_{0}} \frac{\partial {v}_{g}}{\partial x} \qquad ( 3 )$$

and substituting

$$\frac{\partial {v}_{g}}{\partial x}$$

from (1) into (3) gives:

$$\frac{{\partial }^{2} {p}_{g}}{\partial {x}^{2}} = \frac{{ϕ}_{g} η}{{k}_{0} {K}_{g}} \frac{\partial {p}_{g}}{\partial t} \qquad ( 4 )$$

Transposing (4) gives:

$$\frac{\partial {p}_{g}}{\partial t} = \frac{{k}_{0} {K}_{g}}{{ϕ}_{g} η} \frac{{\partial }^{2} {p}_{g}}{\partial {x}^{2}} \qquad ( 5 )$$

which is a diffusion equation of the form:

$$\frac{\partial {p}_{g}}{\partial t} = {h}^{2} \frac{{\partial }^{2} {p}_{g}}{\partial {x}^{2}} \qquad ( 6 )$$

and has the solution:

$${p}_{g} ( x , t ) = {P}_{0} {ⅇ}^{- \sqrt{ω / 2 {h}^{2}} x} sin ( ω t - \sqrt{ω / 2 h} ) \qquad ( 7 )$$

where ω=2πf is the sound frequency in radians per second,

f is the frequency in Hz, and

P₀ is the sound pressure incident on the lung.

Therefore the wave velocity vₗ and attenuation αₗ in the lung tissue are given by:

$$\begin{matrix}{v}_{l} = \sqrt{2 {h}^{2} ω} = \sqrt{\frac{4 π {k}_{0} {K}_{g} f}{{ϕ}_{g} η}} m \text{/} sec & ( 8 ) \\ {α}_{l} = 8.68 \sqrt{ω / 2 {h}^{2}} = 8.68 \sqrt{\frac{π f {ϕ}_{g} η}{{k}_{0} {K}_{g}}} dB \text{/} m & ( 9 )\end{matrix}$$

It can be seen from equations (8) & (9) that if K_{g}, η, and φ_{g} are constant, both velocity and attenuation depend on the acoustic permeability of the lung which increases with pore size. It is also evident that the velocity is highly dispersive, increasing as the square root of sound frequency, as does attenuation.

Finally from (8), we can calculate the velocity dispersion with frequency which is:

$$\frac{ⅆ {v}_{l}}{ⅆ f} = \sqrt{\frac{π {k}_{0} {K}_{g}}{f {ϕ}_{g} η}} \qquad ( 10 )$$

Equation 10 indicates that velocity dispersion is directly proportional to the square root of permeability and inversely proportional to the square root of the frequency. Since the current school of thought indicates that permeability of the lung increases with the progression of emphysema, then velocity dispersion would increase over the entire frequency range with development of the disease, but this change is expected to be progressively smaller with increasing frequency. Thus, it is clear that velocity dispersion increases with acoustic permeability of the lung, attributable to an increase in pore size.

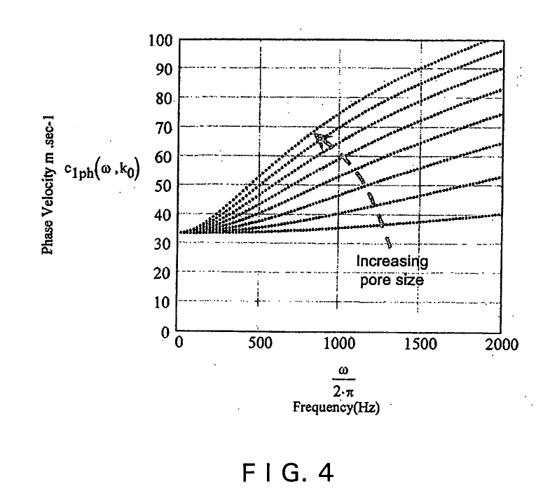

FIG. 4 shows a theoretical graph of frequency versus velocity for various lung permeability (permeability being an acoustic parameter that increases as pore size and pore numbers increase). As can be seen, velocities for individual frequencies increase, as does the dispersion (which can be taken as the gradient of the various permeability plots). It is noted here that the acoustic transmission characteristics, e.g. velocity and velocity dispersion, can vary based on both fenestra sizes and the number of fenestrae present in the lung.

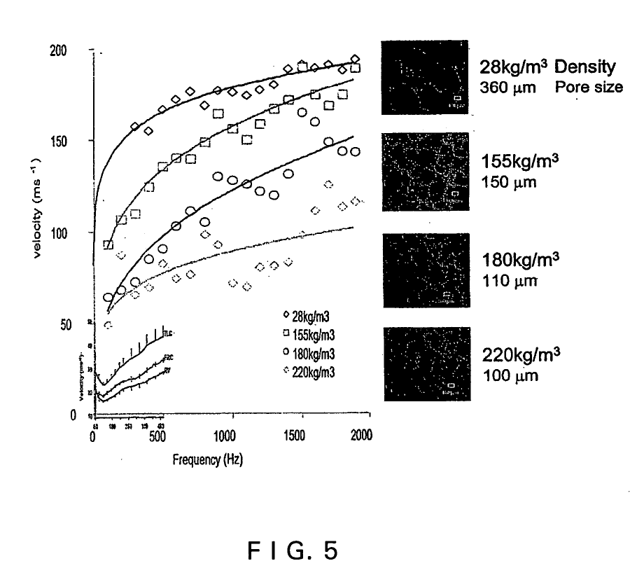

FIG. 5 shows the effects on velocity determined using a model of the lung (i.e., a lung analog), in which the pore sizes, i.e., alveolar fenestrae sizes, are increased. As can be seen, both the velocity and velocity dispersion of an acoustic signal increase with increase in permeability. Latex foam and polyurethane foam among other foam materials may be used as emphysematous lung analogs. FIG. 5 also has superimposed on it plots taken from actual subjects having normal lungs at total lung capacity (TLC), functional residual capacity (FRC) and residual volume (RV). The superimposed data illustrates a distinct separation of velocities which is indicative of the change in acoustic transmission characteristics which occurs during development of the disease, when compared with the acoustic transmission characteristics of a normal lung.

Unlike the prior art systems, the present invention uses the porous microstructure of the lung tissue to determine COPD and a stage thereof. This methodology is particularly well suited to detection, staging and monitoring of emphysema, manifested by a change in the quantity and size of pores (fenestrae) in the lung, causing the lung structure to change from what may be considered a closed cell-type to an open cell-type structure in which porous communication between adjacent alveoli increases. This facilitates detection of very early stage emphysema (i.e., when the fenestrae are still microscopic in size, and the patient is still substantially asymptomatic) which hitherto has not been possible using such a non-invasive, easy to use and economical method and apparatus.

Although the present invention has been described with reference to preferred embodiments, numerous modifications and variations can be made without departing from the scope of the invention. The invention is defined by the appended claims; no other limitation, such as details of the specific preferred embodiments disclosed, is intended or should be inferred.

### REFERENCES

Australian and New Zealand Neonatal Network. Annual Report, 1996-1997.
Baumer J H. International randomized controlled trial of patient triggered ventilation in neonatal respiratory distress syndrome. Arch Dis Child 82: F5-F10, 2000.
Bernstein G, Mannino F L, Heldt G P, Callahan J D, Bull D H, Sola A, Ariagno R L, Hoffman G L, Frantz I D 3rd, Troche B I, Roberts J L, Dela Cruz T V, and Costa E. Randomized multicenter trial comparing synchronized and conventional intermittent mandatory ventilation in neonates. J Pediatr 128: 453-63, 1996.
Brenner M, Hann N M, Mina-Araghi R, Gelb A F, McKenna R J, Colt H. Innovative approaches to lung volume reduction for emphysema. Chest, 126 (1) July 2004, pp. 239-248.
Dreyfuss D, Basset G, Soler P and Saumon G. Intermittent positive-pressure hyperventilation with high inflation pressures produces pulmonary microvascular injury in rats. Am Rev Resp Dis 132: 880-884, 1985.
Fahy, F. (1985) Sound and Structural Vibration. Radiation, Transmission and Response. London: Academic Press.
Froese A B. Role of lung volume in lung injury: HFO in the atelectasis-prone lung. Acta Anaesthesiol Scand Suppl 90:126-130, 1989.
Froese A B. High frequency oscillatory ventilation for adult respiratory distress syndrome: Let's get it right this time! Crit Cae Med 25: 906-908, 1997
Gerstmann D R, Minton S D, Stoddard R A, Meredith K S, Monaco F, Bertrand J M, Battisti O, Langhendries J P, Francois A and Clark R H. The Provo multicenter early high-frequency oscillatory ventilation trial: improved pulmonary and clinical outcome in respiratory distress syndrome Pediatrics. 98: 1044-1057, 1996.
Goncharoff, V., Jacobs, J E, and Cugell, D W Wideband acoustic transmission of human lungs. Med. Biol. Eng. Comp. 27:513-519, 1989.
HIFI Study Group. High frequency oscillatory ventilation compared with conventional mechanical ventilation in the management of respiratory failure in preterm infants. N Engl J Med 320: 88-93, 1989.
Jobe A. Pulmonary surfactant therapy. N Engl J Med 328: 861-864, 1993.
Kraman, S. S. Speed of low-frequency sound through lungs of normal men. J. Appl. Physiol. 55:1862-1867, 1983.
Lowe R D and Robinson B F. A physiological approach to clinical methods. Churchill, London, 1970.
McCulloch, P R, Forkert P G and Froese A B. Lung volume maintenance prevents lung injury during high-frequency oscillatory ventilation in surfactant-deficient rabbits. Am Rev Respir Dis 137: 1185-1192, 1988.
Northway H Q, Rosen R C and Porter D Y. Pulmonary disease following respiratory therapy of hyaline membrane disease. N Engl J Med 276: 357-368, 1967.
Rice, D. A. (1983) Sound speed in pulmonary parenchyma. J. Appl. Physiol. 54:304-308.
Rife D D & Vanderkooy J. Transfer function measurement with maximum length sequences. J Audio Eng Soc 37: 419-444, 1989.
Sheridan, B (2000) Acoustic evaluation of lung inflation in the preterm infant. B. Med. Sci. Thesis, RCBHR, Monash University.
Taghizadeh A & Reynolds E O R. Pathogenesis of bronchopulmonary dysplasia following hyaline membrane disease. Am J Pathol 82: 241-264, 1976.
Wodicka, G. R. and Shannon, D. C. Transfer function of sound transmission in subglottal human respiratory system at low frequencies. J. Appl. Physiol. 69(6):2126-2130, 1990.
Wodicka G R, Stevens, K N, Golub, H L, Cravalho, E G and Shannon, D. C. A model of acoustic transmission in the respiratory system. IEEE Ttrans Biomed. Eng. 36: 925-934, 1989.

What is claimed is:

1. A method for assessing the condition of tissue, comprising the steps of:
(a) introducing sound into tissue with a sound-introducing means;
(b) detecting a portion of the sound with a sound-detecting means after it has passed through at least a portion of the tissue;
(c) determining at least one sound-propagation parameter by comparing at least one property of the introduced sound and at least one property of the detected sound;
(d) identifying a region of examination of the tissue, based in part on either at least one property of the sound-introducing means or at least one property of the sound-detecting means; and
(e) assessing the condition of the tissue in the region of examination, based in part on the sound-propagation parameter.

2. A method according to claim 1, wherein the introduced sound has the majority of its energy within a frequency range from about 100 Hz to about 50 kHz.

3. A method according to claim 1, wherein the introduced sound has the majority of its energy within audible frequencies.

4. A method according to claim 1, wherein the tissue is lung tissue.

5. A method according to claim 1, wherein the introduced sound is selected from the group consisting of: a tone, a pulse, white noise, pseudorandom noise, a sequence of tones, a complex multifrequency waveform, a swept frequency signal, a frequency-modulated signal, and an amplitude-modulated signal.

6. A method according to claim 1, wherein the at least one sound-propagation parameter is selected from the group consisting of: phase delay, phase velocity, group velocity, amplitude, relative amplitude, attenuation, dispersion, the first derivative of amplitude as a function of frequency, and the ratio of amplitude A1/A2, where A1 is a first sound amplitude in one frequency band, and A2 is a second sound amplitude in a second frequency band.

7. A method according to claim 1, wherein the at least one property of the introduced sound is selected from the group consisting of: phase, amplitude, velocity, power, energy, and frequency.

8. A method according to claim 1, wherein the at least one property of the detected sound is selected from the group consisting of: phase, amplitude, velocity, power, energy, and frequency.

9. A method according to claim 1, wherein the at least one property of the sound-introducing means is selected from the group consisting of: spatial location, size, orientation, and shape.

10. A method according to claim 1, wherein the at least one property of the sound-detecting means is selected from the group consisting of: spatial location, size, orientation, and shape.

11. A method according to claim 1, wherein the step of assessing the condition of the tissue comprises first calculating a tissue-condition parameter.

12. A method according to claim 1, wherein the step of introducing sound comprises inserting the sound-introducing means within the tissue.

13. A method according to claim 1, wherein the step of detecting a portion of the sound comprises inserting the sound-detecting means within the tissue.

14. A method according to claim 1, wherein the sound-introducing means is located within the tissue.

15. A method according to claim 1, wherein the sound-detecting means is located within the tissue.

16. A method according to claim 1, wherein the region of examination is in the vicinity of an interventional or diagnostic device that has been positioned within or adjacent to the tissue.

17. A method according to claim 16, wherein the interventional or diagnostic device is selected from the group consisting of: a bronchoscope, a catheter, and an endoscope.

18. A method according to claim 16, wherein the sound-detecting means is attached to the interventional or diagnostic device.

19. A method according to claim 16, wherein the sound-introducing means is attached to the interventional or diagnostic device.

20. A method according to claim 16, wherein the interventional or diagnostic device comprises a reflective device that reflects the introduced sound in the general direction of the sound-detecting means.

21. A method according to claim 20, wherein the reflective device is attached to the interventional or diagnostic device.

22. A method according to claim 11, wherein the tissue-condition parameter is an index indicative of one of the group consisting of: tissue microstructure, airway dimensions, fenestrae size, airway conductance, tissue permeability, tissue permittivity, tissue elasticity, and tissue viscosity.

23. A method according to claim 11, further comprising the step of: deriving information relating to the type or stage of disease in the tissue by comparing the tissue-condition parameter with a library of tissue-condition parameters derived from clinical studies.

24. A device for assessing the condition of tissue, comprising:
(a) means for introducing sound into tissue;
(b) means for detecting a portion of the sound after it has passed through at least a portion of the tissue;
(c) means for determining at least one sound-propagation parameter by comparing at least one property of the introduced sound and at least one property of the detected sound;
(d) means for identifying a region of examination of the tissue, based in part on either at least one property of the sound-introducing means or at least one property of the sound-detecting means; and
(e) means for assessing the condition of the tissue in the region of examination, based in part on the sound-propagation parameter.

25. A device according to claim 24, wherein the introduced sound has the majority of its energy within a frequency range from about 100 Hz to about 50 kHz.

26. A device according to claim 24, wherein the introduced sound has the majority of its energy within audible frequencies.

27. A device according to claim 24, wherein the tissue is lung tissue.

28. A device according to claim 24, wherein the introduced sound is selected from the group consisting of: a tone, a pulse, white noise, pseudorandom noise, a sequence of tones, a complex multifrequency waveform, a swept frequency signal, a frequency-modulated signal, and an amplitude-modulated signal.

29. A device according to claim 24, wherein the at least one sound-propagation parameter is selected from the group consisting of: phase delay, phase velocity, group velocity, amplitude, relative amplitude, attenuation, dispersion, the first derivative of amplitude as a function of frequency, and the ratio of amplitude A1/A2, where A1 is a first sound amplitude in one frequency band, and A2 is a second sound amplitude in a second frequency band.

30. A device according to claim 24, wherein the at least one property of the introduced sound is selected from the group consisting of: phase, amplitude, velocity, power, energy, and frequency.

31. A device according to claim 24, wherein the at least one property of the detected sound is selected from the group consisting of: phase, amplitude, velocity, power, energy, and frequency.

32. A device according to claim 24, wherein the at least one property of the sound-introducing means is selected from the group consisting of: spatial location, size, orientation, and shape.

33. A device according to claim 24, wherein the at least one property of the sound-detecting means is selected from the group consisting of: spatial location, size, orientation, and shape.

34. A device according to claim 24, wherein the assessing means comprises means for calculating a tissue-condition parameter.

35. A device according to claim 24, wherein the sound-introducing means is inserted within the tissue.

36. A device according to claim 24, wherein the sound-detecting means is inserted within the tissue.

37. A device according to claim 24, wherein the sound-introducing means is located within the tissue.

38. A device according to claim 24, wherein the sound-detecting means is located within the tissue.

39. A device according to claim 24, wherein the region of examination is in the vicinity of an interventional or diagnostic device that has been positioned within or adjacent to the tissue.

40. A device according to claim 39, wherein the interventional or diagnostic device is selected from the group consisting of: a bronchoscope, a catheter, and an endoscope.

41. A device according to claim 39, wherein the sound-detecting means is attached to the interventional or diagnostic device.

42. A device according to claim 39, wherein the sound-introducing means is attached to the interventional or diagnostic device.

43. A device according to claim 39, wherein the interventional or diagnostic device comprises a reflective device that reflects the introduced sound in the general direction of the sound-detecting means.

44. A device according to claim 43, wherein the reflective device is attached to the interventional or diagnostic device.

45. A device according to claim 34, wherein the tissue-condition parameter is an index indicative of one of the group consisting of: tissue microstructure, airway dimensions, fenestrae size, airway conductance, tissue permeability, tissue permittivity, tissue elasticity, and tissue viscosity.

46. A device according to claim 34, further comprising: means for deriving information relating to the type or stage of disease in the tissue by comparing the tissue-condition parameter with a library of tissue-condition parameters derived from clinical studies.

47. A device for assessing the condition of tissue, comprising:
(a) a first transducer that introduces sound into tissue;
(b) a second transducer that detects a portion of the sound after it has passed through at least a portion of the tissue;
(c) a sound-propagation comparator that determines at least one sound-propagation parameter by comparing at least one property of the introduced sound and at least one property of the detected sound;
(d) an examination-region identifier that identifies a region of the tissue, based in part on either at least one property of the first transducer or at least one property of the second transducer; and
(e) a tissue-condition assessor that assesses the condition of the tissue in the region of examination, based in part on the sound-propagation parameter.

48. A device according to claim 47, wherein the introduced sound has the majority of its energy within a frequency range from about 100 Hz to about 50 kHz.

49. A device according to claim 47, wherein the introduced sound has the majority of its energy within audible frequencies.

50. A device according to claim 47, wherein the tissue is lung tissue.

51. A device according to claim 47, wherein the introduced sound is selected from the group consisting of: a tone, a pulse, white noise, pseudorandom noise, a sequence of tones, a complex multifrequency waveform, a swept frequency signal, a frequency-modulated signal, and an amplitude-modulated signal.

52. A device according to claim 47, wherein the at least one sound-propagation parameter is selected from the group consisting of: phase delay, phase velocity, group velocity, amplitude, relative amplitude, attenuation, dispersion, the first derivative of amplitude as a function of frequency, and the ratio of amplitude A1/A2, where A1 is a first sound amplitude in one frequency band, and A2 is a second sound amplitude in a second frequency band.

53. A device according to claim 47, wherein the at least one property of the introduced sound is selected from the group consisting of: phase, amplitude, velocity, power, energy, and frequency.

54. A device according to claim 47, wherein the at least one property of the detected sound is selected from the group consisting of: phase, amplitude, velocity, power, energy, and frequency.

55. A device according to claim 47, wherein the at least one property of the first transducer is selected from the group consisting of: spatial location, size, orientation, and shape.

56. A device according to claim 47, wherein the at least one property of the second transducer is selected from the group consisting of: spatial location, size, orientation, and shape.

57. A device according to claim 47, wherein the tissue-condition assessor comprises a parameter calculator that calculates a tissue-condition parameter.

58. A device according to claim 47, wherein the first transducer is inserted within the tissue.

59. A device according to claim 47, wherein the second transducer is inserted within the tissue.

60. A device according to claim 47, wherein the first transducer is located within the tissue.

61. A device according to claim 47, wherein the second transducer is located within the tissue.

62. A device according to claim 47, wherein the region of examination is in the vicinity of an interventional or diagnostic device that has been positioned within or adjacent to the tissue.

63. A device according to claim 62, wherein the interventional or diagnostic device is selected from the group consisting of: a bronchoscope, a catheter, and an endoscope.

64. A device according to claim 62, wherein the first transducer is attached to the interventional or diagnostic device.

65. A device according to claim 62, wherein the second transducer is attached to the interventional or diagnostic device.

66. A device according to claim 62, wherein the interventional or diagnostic device comprises a reflective device that reflects the introduced sound in the general direction of the second transducer.

67. A device according to claim 66, wherein the reflective device is attached to the interventional or diagnostic device.

68. A device according to claim 57, wherein the tissue-condition parameter is an index indicative of one of the group consisting of: tissue microstructure, airway dimensions, fenestrae size, airway conductance, tissue permeability, tissue permittivity, tissue elasticity, and tissue viscosity.

69. A device according to claim 57, further comprising: an information deriver that derives information relating to the type or stage of disease in the tissue by comparing the tissue-condition parameter with a library of tissue-condition parameters derived from clinical studies.
